(19) United States
(12) Patent Application Publication
(10) Pub. No.: US 2008/0182819 A1
(43) Pub. Date: Jul. 31, 2008

(54) CHITOSAN COMPOSITIONS AND METHODS FOR USING CHITOSAN COMPOSITIONS IN THE TREATMENT OF HEALTH DISORDERS

(75) Inventor: Stacy H. Fuchino, Palos Verdes, CA (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: Act III Licensing, LLC, Beverly Hills, CA (US)

(21) Appl. No.: 11/668,383

(22) Filed: Jan. 29, 2007

(51) Int. Cl. A61K 31/722; A61P 25/28

(52) U.S. Cl. 514/55

(57) ABSTRACT

Low molecular weight chitosan compositions may be used in methods for treating a wide variety of mental and/or physical disorders in an animal. The low molecular weight chitosan compositions may be formulated as a capsule for oral administration in treating mental disorders, such as dementia or as a spray for treating physical disorders, such as skin disorders in the animal.

### FIELD OF THE INVENTION

The present invention relates generally to chitosan compositions and methods for using chitosan compositions to treat a variety of health and/or mental disorders in an animal.

### BACKGROUND OF THE INVENTION

Chitosan is a linear polysaccharide composed of randomly distributed β-(1-4)-linked D-glucosamine (deacetylated unit) and N-acetyl-D-glucosamine (acetylated unit). Chitosan is produced by the deacetylation of chitin, which is the structural element in the exoskeleton of crustaceans, such as crabs, lobsters, and shrimp.

The consumption of chitosan is believed to provide certain beneficial health effects, such as weight loss, inhibiting LDL cholesterol and boosting HDL cholesterol. Chitosan is also believed to provide immune enhancement, anti-tumor effects, and anti-bacterial effects.

In spite of its beneficial health effects, the bioavailability of chitosan is limited due to its high molecular mass, high viscosity and, thus, low absorption for in vivo applications. Thus, recent studies on chitosan depolymerization have drawn considerable attention, since the products obtained are easily water-soluble and are also believed to possess versatile biofunctional properties.

There are methods to produce low molecular weight chitosan of lower viscosity and higher solubility. For example, low molecular weight chitosan may be produced by treating chitosan with 0.007 to 0.35% hydrogen peroxide solution adjusted to a pH of about 6 to about 8. Chitosan oligosaccharides may also be prepared and/or obtained by an enzymatic process that is described in U.S. Pat. No. 5,482,843.

### BRIEF SUMMARY OF THE PREFERRED EMBODIMENTS

Chitosan compositions and methods for using chitosan compositions in the treatment of heath disorders in an animal are disclosed.

In one preferred embodiment, methods for treating mental disorder in an animal are provided. The method comprising administering an effective amount of a low molecular weight chitosan composition to the animal.

In another preferred embodiment, methods for treating a physical disorder in an animal are provided. The methods comprise spraying a low molecular weight chitosan composition on the affected area of the animal.

In a further preferred embodiment, a chitosan spray comprising low molecular weight chitosan is provided.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### DETAILED DESCRIPTION OF THE PREFERRED EMBODIMENTS

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

Low molecular weight chitosan has been discovered to have numerous beneficial effects on the health of animals, such as providing immune enhancement, anti-tumor effects, antibacterial function, wound healing, improvements in skin disorders, sugar regulation, and liver and kidney enhancement, to name a few.

Low molecular weight chitosan has also been discovered to have significant beneficial effects on animals suffering from mental disorders, such as dementia. Improvements in the animal's mental health has been clinically observed with the administration of chitosan over a period of 4 weeks or more.

The low molecular weight chitosan used in connection with the compositions and methods disclosed herein may be a pharmaceutically acceptable salt form or derivatives thereof. In a particularly preferred embodiment, the chitosan is a low molecular weight chitosan having a molecular weight less than about 8,000 Da, preferably less than 5,000 Da, and more preferably less than about 2,500 Da.

The low molecular weight chitosan composition may be formulated in any number of ways depending on the manner in which it is administered to the animal. For example, the low molecular weight chitosan composition may be administered orally, mucosally, and topically, in dosage unit formulations containing conventional pharmaceutically acceptable carriers, adjuvants, and vehicles.

In one preferred embodiment, the composition may be formulated for oral administration as tablets, capsules, granules, powders, syrups, emulsions, suspensions, liquids, and solutions.

In one aspect of the preferred embodiment, the composition is used in the form of tablets or capsules by mixing the low molecular-weight chitosan with a conventional carrier, diluent, binder, and or stabilizer, if necessary. The tablets or capsules can further include lactic acid, sucrose ester, calcium phosphate, L-valine, wax, dextrin, etc.

In another aspect of the preferred embodiment, the composition may be formulated as an aqueous suspension and/or emulsion. If desired, certain sweetening and/or flavoring and/or coloring agents may be added.

In another preferred embodiment, the composition may be formulated for topical or transdermal administration, such as a cream, lotion, spray, or provided in connection with a transdermal patch.

In one aspect of this preferred embodiment, the composition is an aqueous solution and provided in the form of a spray for topical administration on the animal. The composition may be a solution comprising from about 1% to about 50% by weight, preferably from about 5% to about 25% by weight, and more preferably about 8% to about 15% by weight of the low molecular weight chitosan in water. Carrier compounds may be included, such as starch.

The amount and frequency of administering the low molecular weight chitosan composition vary according to a number of factors, including the age, weight, and medical condition, the type and severity of the mental disorder to be treated, the route and frequency of administration, and the route and frequency of administration of the animal.

Thus, the dosage regimen may vary widely, but can be determined routinely using standard methods. A daily dose of about 1 to 50 mg/kg body weight, preferably from about 2.5 to 25 mg/kg body weight, and more preferably from about 5 to 15 mg/kg body weight may be appropriate. The daily dosage can be administered in one to four doses per day.

In one preferred embodiment, the low molecular weight chitosan is administered orally in a dose of about 1 mg to about 2,500 mg per day, preferably from about 15 mg to about 1,000 mg per day, and more preferably from about 25 mg to about 500 mg per day.

In another preferred embodiment, the low molecular weight chitosan is administered topically by a solution comprising from about 1% to about 50% by weight, preferably from about 5% to about 25% by weight, and more preferably about 8% to about 15% by weight of the low molecular weight chitosan in water. Carrier compounds may be included, such as starch

### EXAMPLE 1

### Preparation and Use of Chitosan Spray Composition to Treat Dermatological Conditions

A liquid composition comprising low molecular weight chitosan and water was prepared and placed in a spray container. The spray may be actuated by a pump or by an aerosol.

Application of the chitosan liquid composition by spraying has been found to ameliorate various dermatological conditions in animals, such has “hot spots”, surgical incisions, itchy skin, and post surgery applications. For example, cats are known to have mouth lesions. Application of the liquid chitosan composition has been shown to improve this condition.

### EXAMPLE 2

### Preparation and Use of Chitosan Spray Capsules to Treat Dementia in Dogs and Cats

Low molecular weight chitosan compositions may be provided in dry capsule form by mixing the low molecular-weight chitosan with a conventional carrier, diluent, binder, and or stabilizer, if necessary. The capsules can further include lactic acid, sucrose ester, calcium phosphate, L-valine, wax, dextrin, etc.

The amount of low molecular weight chitosan contained in the capsule depends on a variety of factors, including age, weight, and medical condition, the type and severity of the mental disorder to be treated, and the frequency of administration.

It has been found that the administration of 25 mg of the low molecular weight chitosan to a small dog weighing approximately 10-11 pounds for a duration of 2 weeks has been effective in reducing the symptoms of dementia. Table 1 shows the dosage and duration for the administration of the chitosan compositions for cats based on their weight.

TABLE 1
Chitosan dosage and duration for Cats

| Cat (weight in lbs.) | Chitosan Dosage | Duration |
|---|---|---|
| Small 5–10 lbs | 25 mg/day | 4 weeks |
| Large 11–20 lbs | 50 mg/day | 4 weeks |

Table 2 shows the dosage and duration for the administration of the chitosan compositions for dogs based on their weight.

TABLE 2
Chitosan dosage and duration for Dogs

| Dog (weight in lbs.) | Chitosan Dosage | Duration |
|---|---|---|
| Small 5–15 lbs | 100 mg/day | 4 weeks |
| Small–Medium 16–40 lbs | 200 mg/day | 4 weeks |
| Medium–Large 41–60 lbs | 300 mg/day | 4 weeks |
| Large 61–80 lbs | 400 mg/day | 4 weeks |
| Extra large >81 lbs | 500 mg/day | 4 weeks |

The invention described and claimed herein is not to be limited in scope by the specific preferred embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims

1. A method of treating mental disorder in an animal, the method comprising administering an effective amount of a low molecular weight chitosan composition to the animal.

2. The method of claim 1, wherein the metal disorder is dementia.

3. The method of claim 1, wherein the low molecular weight chitosan has a molecular weight of about 5,000 to about 8,000 Da.

4. The method of claim 1, wherein the low molecular weight chitosan as a molecular weight of about 5,000 Da or less.

5. The method of claim 4, wherein the low molecular weight chitosan composition is administered orally.

6. The method of claim 4, wherein the low molecular weight chitosan composition is administered mucosally.

7. The method of claim 5, wherein the low molecular weight chitosan composition is formulated in any one or more of the following forms: tablet, capsules, granules, powders, syrups, emulsions suspensions, liquids, and solutions.

8. The method of claim 7, wherein the low molecular weight chitosan composition is formulated in a capsule.

9. The method of claim 8, wherein the effective amount of the low molecular chitosan composition administered to the animal is about 1 to about 50 mg/kg body weight of the animal.

10. The method of claim 8, wherein the effective amount of the low molecular chitosan composition administered to the animal is about 2.5 to about 25 mg/kg body weight of the animal.

11. The method of claim 8, wherein the effective amount of the low molecular chitosan composition administered to the animal is about 5 to about 15 mg/kg body weight of the animal.

12. The method of claim 8, wherein the animal to be treated is a cat.

13. The method of claim 12, wherein the cat weighs about 5 to about 10 lbs and the effective amount of a low molecular weight chitosan composition to the cat is about 25 mg/day for duration of 4 weeks.

14. The method of claim 12, wherein the cat weighs about 11 to about 20 lbs and the effective amount of a low molecular weight chitosan composition to the cat is about 50 mg/day for duration of 4 weeks.

15. The method of claim 8, wherein the animal to be treated is a dog.

16. The method of claim 15, wherein the dog weighs about 5 to about 15 lbs and the effective amount of a low molecular weight chitosan composition to the dog is about 100 mg/day for duration of 4 weeks.

17. The method of claim 15, wherein the dog weighs about 16 to about 40 lbs and the effective amount of a low molecular weight chitosan composition to the dog is about 200 mg/day for duration of 4 weeks.

18. The method of claim 15, wherein the dog weighs about 41 to about 60 lbs and the effective amount of a low molecular weight chitosan composition to the dog is about 300 mg/day for duration of 4 weeks.

19. The method of claim 15, wherein the dog weighs about 61 to about 80 lbs and the effective amount of a low molecular weight chitosan composition to the dog is about 400 mg/day for duration of 4 weeks.

20. The method of claim 15, wherein the dog weighs over 80 lbs and the effective amount of a low molecular weight chitosan composition to the dog is about 500 mg/day for duration of 4 weeks.

21. A method of treating a physical disorder in an animal by spraying a low molecular weight chitosan composition on the affected area.

22. The method of claim 21, wherein the low molecular weight chitosan has a molecular weight of about 5,001 to about 8,000 Da.

23. The method of claim 21, wherein the low molecular weight chitosan as a molecular weight of about 5,000 Da or less.

24. The method of claim 23, wherein the low molecular weight chitosan composition is administered topically.

25. The method of claim 24, wherein the low molecular weight chitosan composition is formulated for topical administration in any one or more of the following: cream, lotion, spray, and transdermal patch.

26. The method of claim 25, wherein the low molecular weight chitosan composition is formulated in a spray solution.

27. The method of claim 26, wherein the spray solution comprises from about 1-50% by weight of the low molecular chitosan in water.

28. The method of claim 27, wherein the spray solution comprises from about 5-25% by weight of the low molecular chitosan in water.

29. The method of claim 28, wherein the spray solution comprises from about 8-15% by weight of the low molecular chitosan in water.

30. A chitosan composition for administration as a spray, the chitosan composition comprising low molecular weight chitosan and water.

31. The chitosan composition of claim 30, wherein the low molecular weight chitosan has a molecular weight of about 5,001 to about 8,000 Da.

32. The chitosan composition of claim 30, wherein the low molecular weight chitosan has a molecular weight of about 5,000 Da or less.

33. The method of claim 32, wherein the spray solution comprises from about 1-50% by weight of the low molecular chitosan in water.

34. The method of claim 33, wherein the spray solution comprises from about 5-25% by weight of the low molecular chitosan in water.

35. The method of claim 34, wherein the spray solution comprises from about 8-15% by weight of the low molecular chitosan in water.
